(19) United States
(12) Patent Application Publication
(10) Pub. No.: US 2004/0230098 A1
(43) Pub. Date: Nov. 18, 2004

(54) Endoscope illumination system

(75) Inventors: Richard A. Farkas, Bloomfield Hills, MI (US); Terrance R. Boyd, Jackson, MI (US); Richard L. Fisher, Ann Arbor, MI (US); Albert J. Boehnlein, Ypsilanti, MI (US)

Correspondence Address: GIFFORD, KRASS, GROH, SPRINKLE, ANDERSON & CITKOWSKI, PC, 280 N OLD WOODARD AVE, SUITE 400, BIRMINGHAM, MI, 48009, US

(73) Assignee: Inner Vision Imaging, L.L.C., Farmington Hills, MI

(21) Appl. No.: 10/437,743

(22) Filed: May 14, 2003

(51) Int. Cl. A61B 1/06

(52) U.S. Cl. 600/178

(57) ABSTRACT

An endoscope illumination system for use with an endoscope having both a microscopic mode and a macroscopic mode. The illumination system includes a source of broadband light and includes a source of laser illumination, which may be infrared illumination. A circuit determines the mode of operation of the endoscope. This circuit activates the source of broadband light and deactivates the source of laser illumination when the endoscope is in its macroscopic mode. Conversely, the circuit deactivates the source of broadband light and activates the source of laser illumination when the endoscope is in the microscopic mode.

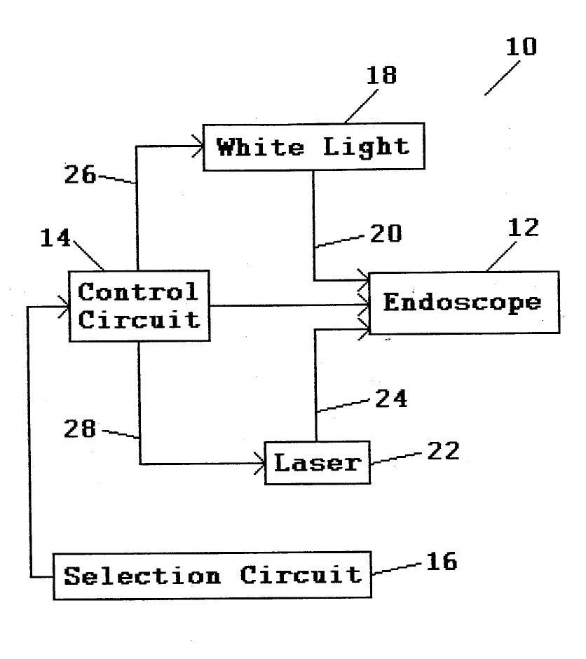

### BACKGROUND OF THE INVENTION

I. Field of the Invention

The present invention relates to an illumination system for use with an endoscope.

II. Description of Related Art

There are endoscopes which are operational in both a microscopic mode as well as a macroscopic mode. One such endoscope is disclosed in U.S. Pat. No. 6,530,882, entitled “Endoscope Having Microscopic and Macroscopic Magnification” and which issued on Mar. 11, 2003.

For use in a macroscopic mode, a broadband light source, such as a xenon light source, is used to illuminate the body cavity for viewing through the endoscope. Conversely, laser illumination is conventionally used for illuminating the body cavity when the endoscope is utilized in the microscopic mode. Such laser illumination may be in the infrared frequency range for better tissue penetration when in the microscopic mode.

Conventionally, during the use of the endoscope, it has been necessary to manually switch the broadband light sources and laser source. Consequently, as the endoscope is switched between the microscopic and macroscopic mode, the illumination would be manually selected between the broadband or white light and laser illumination for the desired imaging.

In practice, both the broadband light source and the laser source have a limited number of power-on hours before replacement is required. Furthermore, both the broadband light source and laser are relatively expensive and inconvenient to replace.

Illumination by the white light source during the microscopic mode also may cause stray white light in the system, which is undesirable when in the microscopic mode. Additionally, when the endoscope is in the microscopic mode, energy is wasted by operating the white light source.

### SUMMARY OF THE PRESENT INVENTION

The present invention provides an endoscope illumination system for use with an endoscope having both a microscopic mode and a macroscopic mode which overcomes all of the above-mentioned disadvantages of the previously known devices.

In brief, the present invention includes a broadband light source, typically a white light source. Preferably, the broadband light source comprises a xenon light bulb, although other white light sources may alternatively be employed. Such other sources include halogen light sources, light emitting diodes and arrays of diodes, both white and colored LEDs. Conventional light conveying means, typically fiber optics, mirrors and the like, are employed to convey the broadband light from the light source and to the endoscope.

The illumination system further includes a source of laser illumination. This laser illumination may be invisible light, preferably in the infrared range, for better tissue penetration. The source of laser illumination is conveyed to the endoscope through any conventional means, such as an optical fiber and the like.

A control circuit is operatively coupled to the endoscope and determines which mode, i.e. either microscopic mode or macroscopic mode, has been selected by the operator of the endoscope. When the macroscopic mode has been selected, the control circuit generates an output signal to activate or turn on the broadband light source and simultaneously sends a further signal to the laser illumination source to deactivate or turn off the source of laser illumination. Conversely, when the endoscope is in its microscopic mode, the control circuit generates an output signal to deactivate or turn off the broadband light source and, simultaneously, generates an output signal to activate or turn on the source of laser illumination.

Consequently, the illumination system of the present invention activates or turns on the broadband light source and the source of laser illumination in a mutually exclusive fashion. By doing so, the effective lives of the broadband light source and laser source are both extended and power is conserved. Additionally, by turning off the broadband light source when in the microscopic mode, the possibility of stray broadband light within the imaging system is eliminated.

### BRIEF DESCRIPTION OF THE DRAWING

A better understanding of the present invention will be had upon reference to the following detailed description, when read in conjunction with the accompanying drawing, which is a block diagrammatic view of a preferred embodiment of the endoscope illumination system of the present invention.

### DETAILED DESCRIPTION OF A PREFERRED EMBODIMENT OF THE PRESENT INVENTION

With reference to the drawing, a preferred embodiment of the endoscope illumination system 10 of the present invention is there shown in block diagrammatic form. The illumination system 10 is designed for use with an endoscope 12 which is operable in either a macroscopic mode or a microscopic mode. As used in this application, the term “macroscopic mode” provides low magnification of the area under examination by the endoscope. Such low magnification is typically less than ten times magnification.

Conversely, as used in this patent, the term “microscopic mode” means a sufficiently high magnification to examine body tissues on a cellular level. The endoscope 12, when in its microscopic mode, can be used for a variety of purposes, including examination of tissues for evidence of cancer.

A control circuit 14 is operatively coupled to the endoscope and performs a variety of control functions. For example, the control circuit 14 determines whether the endoscope 12 is in its microscopic mode or macroscopic mode. Such switching of the endoscope 12 between its microscopic mode and its macroscopic mode is typically done in response to a user selection input circuit 16.

A source of broadband light 18 is optically coupled to the endoscope in any conventional fashion, such as by an optical fiber 20. The broadband light source 18 preferably comprises a xenon light bulb but may optionally be any other source of visible illumination such as a halogen light, white LEDs or arrays of white LEDs, colored LEDs or arrays of colored LEDs, and the like. Furthermore, the term “broadband” is used in a generic sense to include any and all types of visible noncoherent illumination which may be used with the endoscope 12.

Similarly, a source of laser illumination 22 is optically coupled to the endoscope 12 by any conventional means, such as an optical fiber 24. The laser 12, furthermore, may be any conventional type of laser, but preferably emits coherent light at a nonvisible wavelength. Preferably, the laser 22 utilizes infrared radiation for better tissue penetration of the imaged tissue when the endoscope is in its microscopic mode.

The control circuit 14 includes a first control line 26 which is operatively coupled to the broadband light source 18 to either activate, i.e. turn on, the broadband light source 18 or to deactivate, i.e. turn off, the broadband light source 18 depending upon the output signal on the control line 26. Similarly, a control line 28 is provided from the control circuit 14 to the laser 22. The control circuit selectively activates, i.e. turns on, or deactivates, i.e. turns off, the laser 22 in dependence upon the signal on the control line 28. Consequently, the control circuit 14 selectively activates and deactivates both the broadband light source 18 and laser 22.

In operation, when the endoscope 12 is in its macroscopic mode, the control circuit 18 generates output signals on its control lines 26 and 28 to activate the broadband light source 18 and substantially simultaneously deactivate the laser 22. Conversely, when the endoscope 12 is switched to its microscopic mode, the control circuit 14 generates output signals on its control lines 26 and 28 to deactivate the broadband light source 18 and substantially simultaneously activate the laser 22. Consequently, the broadband light source 18 and laser 22 are operated in a substantially mutually exclusive fashion depending upon the operating mode of the endoscope 12.

By operating the broadband light source 18 and laser 22 in a mutually exclusive fashion, the present invention effectively extends the life of the white light source and laser source and conserves power. Furthermore, since the broadband light source 18 is deactivated during the microscopic mode, the possibility of stray white light in the optic systems while in the microscopic mode is eliminated.

Having described our invention, however, many modifications thereto will become apparent to those skilled in the art to which it pertains without deviation from the spirit of the invention as defined by the scope of the appended claims.

We claim:

1. An endoscope illumination system for use with an endoscope having a microscopic mode and a macroscopic mode comprising:
a source of broadband light,
a source of laser illumination,
a circuit which determines the mode of operation of the endoscope, said circuit activating said source of broadband light and deactivating said source of laser illumination when the endoscope is in the macroscopic mode, and said circuit deactivating said source of broadband light and activating said source of laser illumination when the endoscope is in the microscopic mode.

2. The invention as defined in claim 1 wherein said source of broadband light comprises a xenon light source.

3. The invention as defined in claim 1 wherein said source of laser illumination emits radiation at a non-visible frequency.

4. The invention as defined in claim 2 wherein said source of laser illumination emits infrared radiation.

5. The invention as defined in claim 1 wherein said source of broadband light comprises a halogen light source.

6. The invention as defined in claim 1 wherein said source of broadband light comprises a white light emitting diode.

7. The invention as defined in claim 6 wherein said source of broadband light comprises an array of white light emitting diodes.

8. The invention as defined in claim 1 wherein said source of broadband light comprises a light emitting diode.

9. The invention as defined in claim 8 wherein said source of broadband light comprises an array of light emitting diodes.

10. The invention as defined in claim 9 wherein said array of light emitting diodes comprises at least two different colored light emitting diodes.
